(19) United States
(12) Patent Application Publication
(10) Pub. No.: US 2009/0131474 A1
(43) Pub. Date: May 21, 2009

(54) SCREENING METHODS FOR PROTEIN KINASE B INHIBITORS EMPLOYING VIRTUAL DOCKING APPROACHES AND COMPOUNDS AND COMPOSITIONS DISCOVERED THEREBY

(76) Inventors: Martino Forino, La Jolla, CA (US); Dawoon Jung, La Jolla, CA (US); Maurizio Pellecchia, La Jolla, CA (US)

Correspondence Address: CATALYST LAW GROUP, APC, 9710 SCRANTON ROAD, SUITE S-170, SAN DIEGO, CA, 92121, US

(21) Appl. No.: 11/817,764

(22) PCT Filed: Mar. 2, 2006
(86) PCT No.: PCT/US2006/007730

§ 371 Date: Apr. 25, 2008

Related U.S. Application Data
(60) Provisional application No. 60/658,828, filed on Mar. 3, 2005.

(51) Int. Cl. A61K 31/47; C12Q 1/48; A61K 31/34; G06F 17/50

(52) U.S. Cl. 514/312; 435/15; 514/313; 514/461; 703/1

(57) ABSTRACT

The present invention describes an improved method for screening compounds for activity in inhibiting the enzymatic activity of Akt1 protein kinase, also known as Protein Kinase B, an enzyme that is believed to play a key role in the inhibition of apoptosis and thus in the etiology of cancer and other conditions, including neurodegenerative diseases. In general, the method comprises: (1) providing a plurality of compounds suspected of having Akt1 kinase inhibitory activity; (2) modeling the docking of each of the plurality of the compounds with a target binding site derived from the crystal structure of a ternary complex involving Akt1, a nonhydrolyzable ATP analogue, and a peptide substrate derived from a physiological AKT substrate such that the protein active site is defined including those residues within a defined distance from the nonhydrolyzable ATP analogue; (3) ranking the docked compounds by goodness of fit; (4) further selecting compounds from compounds high ranked by goodness of fit in docking by using one or more screening criteria; (5) optionally, visually analyzing structures of compounds selected in step (4) to remove any compounds with improbable docking geometry; and (6) experimentally testing the selected compounds from step (4) or step (5), if step (5) is performed, to determine their inhibitory activity against Akt1 in order to select compounds with Akt1 inhibitory activity. The invention also encompasses pharmaceutical compositions including compounds whose inhibitory activity against Akt1 is discovered by the screening method, as well as methods of use of the pharmaceutical compositions to treat cancer and other conditions.

### RELATED APPLICATIONS

Benefit of priority under 35 U.S.C. 119(e) is claimed herein to U.S. Provisional Application No. 60/658,828, filed Mar. 3, 2005. The disclosure of the above referenced application is incorporated by reference in its entirety herein.

### FIELD OF THE INVENTION

This application is directed to screening methods for Protein Kinase B inhibitors, particularly screening methods employing virtual docking approaches, and compounds and compositions discovered by the use of these docking methods.

### BACKGROUND OF THE INVENTION

Protein phosphorylation plays a central role in many cellular events such as proliferation, differentiation, survival, and angiogenesis (1). Consequently, unregulated kinase activity can result in uncontrolled cellular growth and inappropriate regulation of apoptosis, which is a key mechanism in oncogenesis suppression (2).

Within this scenario Akt, also known as protein kinase B (PKB), has recently caught scientists' attention, since its aberrant activation has been recognized to be responsible for a wide range of proliferative and antiapoptotic processes in many human tumors (3). Akt is a subfamily consisting of three different cellular isoforms, namely, Akt1 (PKBR), Akt2 (PKBβ), and Akt3 (PKBγ). Akt1 is mostly involved in breast cancer and in gastric adenocarcinomas; Akt2 is amplified in ovarian, pancreatic, and breast cancers; and Akt3 is amplified in breast cancer and prostate cell lines (4).

Akt1 is composed of a kinase domain, a N-terminal pleckstrin homology (PH) domain, and a short carboxyterminal tail region. This protein is activated when Thr308 and Ser473 are phosphorylated (5). Once activated, Akt1 inhibits apoptosis and stimulates cell cycle progression by phosphorylating numerous targets in various cell types, including cancer cells. Consequently, the development of molecules capable of blocking protein kinase B activity is a valuable route for anticancer drug discovery (6, 7, 8, 9).

One of the proteins phosphorylated by activated Akt1 is the protein known as BAD, which normally encourages cells to undergo programmed cell death, or apoptosis. Once phosphorylated, BAD binds to a cytosolic protein designated 14-3-3, which inactivates BAD. Akt1 also promotes cell survival by inhibiting other cell death activators; one route for accomplishing this is by inhibition of transcription of the genes encoding the cell death activators, such as those of the Forkhead family, which are gene regulatory proteins that stimulate the transcription of genes that encode proteins that promote apoptosis.

Because of the importance of this pathway in cancer, as well as in other diseases that involve disruption of normal apoptotic processes, including neurodegenerative conditions, there is a need for improved screening methods for the discovery of inhibitors of Akt1, as well as for compounds and compositions discovered by such screening methods.

### SUMMARY

One aspect of the invention is a screening method that meets these needs and provides efficient, high throughput screening of compounds for Akt1 inhibitory activity. In general, this screening method comprises:

(1) providing a plurality of compounds suspected of having Akt1 kinase inhibitory activity;

(2) modeling the docking of each of the plurality of the compounds with a target binding site derived from the crystal structure of a ternary complex involving Akt1, a nonhydrolyzable ATP analogue, and a peptide substrate derived from a physiological AKT substrate such that the protein active site is defined including those residues within a defined distance from the nonhydrolyzable ATP analogue;

(3) ranking the docked compounds by goodness of fit;

(4) further selecting compounds from compounds high ranked by goodness of fit in docking by using one or more screening criteria;

(5) optionally, visually analyzing structures of compounds selected in step (4) to remove any compounds with improbable docking geometry; and

(6) experimentally testing the selected compounds from step (4) or step (5), if step (5) is performed, to determine their inhibitory activity against Akt1 in order to select compounds with Akt1 inhibitory activity.

Typically, the nonhydrolyzable ATP analogue is AMP-PNP. Typically, the peptide substrate is a peptide substrate derived from GSK-3β.

Typically, the defined distance from the nonhydrolyzable analogue is from about 6.0 Å to about 7.0 Å. Preferably, the defined distance from the nonhydrolyzable analogue is about 6.5 Å.

Typically, the modeling of docking is performed using a docking algorithm. Preferably, the docking algorithm is FlexX.

Typically, the step of further selecting compounds from compounds high ranked by goodness of fit in docking by using one or more screening criteria is performed by using one or more of CSCORE (SYBYL), Drugscore, Goldscore, Chemscore, and GOLD.

Preferably, when the docking algorithm is FlexX, the step of further selecting compounds from compounds high ranked by goodness of fit in docking by using one or more screening criteria is performed by first using Drugscore, and then evaluating and ranking the top docked structures according to Goldscore and Chemscore individually. More preferably, compounds that are highly ranked according to both Goldscore and Chemscore functions, when those are applied individually, are then selected for visual analysis to remove compounds with improbable docking geometries.

Typically, the step of experimentally testing the compounds that emerge from screening in step (4) or step (5), if performed, is performed by testing the compounds at a concentration up to 30 μM. More typically, the concentration is 10 μM.

Typically, compounds screened as positive are capable of binding specifically within the catalytic site of the ATP. Typically, compounds screened as positive act as competitive inhibitors of Akt1, competing with ATP. Typically, compounds screened as positive are involved in hydrogen-bonding interactions with residues Lys181, Ala232, Thr292, and Thr162 of Akt1.

The method can further comprise an additional screening step of measuring a consensus between scoring patterns and hydrogen bonding patterns substantially similar to that observed in the crystal structure of Akt1 in complex with AMP-PMP and selecting compounds that exhibit both highly ranked scoring patterns and hydrogen bonding patterns substantially similar to that observed in the crystal structure of Akt1 in complex with AMP-PMP.

Another aspect of the invention is a method of derivatizing a compound determined to have inhibitory activity against Akt1 kinase to improve its inhibitory activity comprising the steps of:

(1) providing a compound having inhibitory activity against Akt1 kinase;

(2) derivatizing the compound by introducing at least one covalent modification thereto to produce at least one derivative; and

(3) screening the derivatives produced in step (2) for inhibitory activity against Akt1 kinase; and

(4) selecting a derivative that has improved inhibitory activity against Akt1 kinase as compared with the compound provided in step (1).

The step of derivatizing typically comprises at least one reaction selected from the group consisting of the substitution of halogens for one or more hydrogens; the replacement of halogens by hydrogens; the placement, removal or repositioning of carboxyl groups on aromatic rings; the conversion of carboxylic acids into esters and vice versa; the conversion of alcohols into ethers; the substitution of hydrogens on amine groups with alkyl groups; and the removal of alkyl groups on amine groups.

Another aspect of the invention is a pharmaceutical composition for inhibiting Akt 1 kinase comprising:

(a) a compound whose activity in inhibiting Akt1 kinase was discovered by the screening method of the present invention in a quantity sufficient to inhibit Akt1 kinase; and

(b) a pharmaceutically acceptable carrier.

Yet another aspect of the invention is a method of treating a disease or condition characterized by dysregulation of apoptosis comprising administering an effective quantity of the pharmaceutical composition according to the present invention to a subject diagnosed with or suspected of having a disease or condition characterized by dysregulation of apoptosis in order to normalize apoptosis. The disease or condition can be cancer or another condition, such as a neurodegenerative condition.

### BRIEF DESCRIPTION OF THE DRAWINGS

The following invention will become better understood with reference to the specification, appended claims, and accompanying drawings, where:

FIG. 1 is a schematic representation of the virtual docking approaches adopted: (A) an approach involving docking of 50,000 compounds and ranking according to the software FlexX, then ranking the top scoring 2000 compounds with other scoring functions using CSCORE, as well as selecting top ranking compounds with Drugscore, Goldscore, and Chemscore, as well as docking the FlexX top 4000 compounds using GOLD; followed by experimental testing; (B) an approach selecting the top 4000 compounds out of 50,000 docket compounds using FlexX and Drugscore; the top 4000 docked structures were then evaluated and ranked according to Goldscore and Chemscore functions (CSCORE); a list of common 200 compounds was then selected among ranked top 700 compounds according to both scoring functions, and elimination of structures with improbable docking geometry by visual analysis, followed by experimental testing of the remaining 100 compounds.

FIG. 2 is a series of graphs showing the assay of Akt1 inhibition for Compounds 1 and 2: (A) IC₅₀ evaluation for Compound 1; (B) IC₅₀ evaluation for Compound 2; (C) Lineweaver-Burk Km and Km(app) evaluation for Akt1; (D) Akt1 inhibition assay using GSK-3 as a substrate, showing a comparison of Compound 1 and Compound 2 with H89 at 10 μM using an immunological approach after polyacrylamide gel electrophoresis and transfer to a nitrocellulose membrane with rabbit polyclonal anti-phospho-GSK-3α/β (Ser21/9); and (E) dose response for Compound 1.

FIG. 3 shows docking models: (A-C), docked structures of Compounds 1-3 into the ATP binding site of Akt1; (D) hydrogen-bonds between Compound 1 and amino acid residues present in the Akt1 catalytic pocket.

### DETAILED DESCRIPTION OF THE INVENTION

One aspect of the invention is a method of screening compounds for inhibition of Akt1 kinase activity comprising the steps of:

(1) providing a plurality of compounds suspected of having Akt1 kinase inhibitory activity;

(2) modeling the docking of each of the plurality of the compounds with a target binding site derived from the crystal structure of a ternary complex involving Akt1, a nonhydrolyzable ATP analogue, and a peptide substrate derived from a physiological AKT substrate such that the protein active site is defined including those residues within a defined distance from the nonhydrolyzable ATP analogue;

(3) ranking the docked compounds by goodness of fit;

(4) further selecting compounds from compounds high ranked by goodness of fit in docking by using one or more screening criteria; and

(5) optionally, visually analyzing structures of compounds selected in step (4) to remove any compounds with improbable docking geometry; and

(6) experimentally testing the selected compounds from step (4) or step (5), if step (5) is performed, to determine their inhibitory activity against Akt1 in order to select compounds with Akt1 inhibitory activity.

Typically, the nonhydrolyzable ATP analogue is AMP-PNP. Typically, the peptide substrate is a peptide substrate derived from GSK-3β.

Typically, the defined distance from the nonhydrolyzable analogue is from about 6.0 Å to about 7.0 Å. Preferably, the defined distance from the nonhydrolyzable analogue is about 6.5 Å.

Typically, the modeling of docking is performed using a docking algorithm. A particularly preferred docking algorithm is FlexX (BiosolveIT, Sankt Augustin, Germany), but others are known in the art.

The step of further selecting compounds from compounds high ranked by goodness of fit in docking by using one or more screening criteria can employ various screening criteria known in the art, or combinations of those screening criteria. For example, screening can be accomplished using CSCORE (SYBYL) (14), Drugscore (15), Goldscore (16), Chemscore (17), or GOLD (18). These screening methods can be applied sequentially, so that compounds that are high ranked by one screening method can then be rescreened with a second method, and compounds ranked high in both screening methods are selected for further analysis. In one particularly preferred approach, compounds are selected using FlexX and Drugscore, and the top docked structures are evaluated and ranked according to Goldscore and Chemscore functions individually. Compounds that are highly ranked according to both Goldscore and Chemscore functions, when those are applied individually, are then selected for visual analysis to remove compounds with improbable docking geometries.

The molecular parameters that govern binding of substrates and inhibitors to enzymes are well known in the art. Typically, binding is governed by hydrogen bonding, hydrophobic interactions, ionic bonds (salt links), covalent bonds (at certain stages of the reaction), and Van der Waals forces; binding typically involves either a “lock and key” mechanism or an “induced fit” mechanism. These can be modeled by means of appropriate software, taking into account the variation in the strength of the interaction with the distance between the two molecules and that there are six degrees of rotational and translational freedom of one molecule relative to the other as well as the conformational degrees of freedom of each molecule.

Typically, the step of experimentally testing the compounds that emerge from screening in step (4) or step (5), if applicable, are tested at 10 μM or at concentrations up to 30 μM for their Akt1 inhibitory activity. Typically, inhibitory activity is evaluated for the selected compounds by using the Z′-LYTE kit assay provided by Invitrogen Corporation (19).

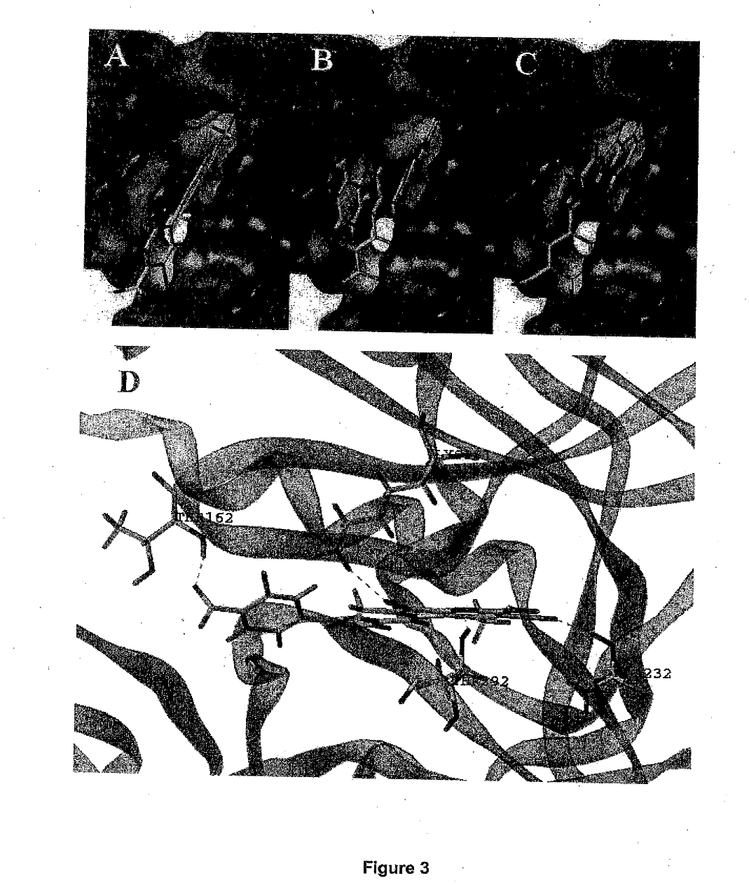

Typically, compounds screened as positive are capable of binding specifically within the catalytic site of the ATP, resembling the binding of the adenosine moiety of this cofactor (FIGS. 3A-C). Kinetic analysis establishes that these compounds act as typical competitive inhibitors; they compete with ATP for binding by the kinase. Accordingly, they affect the Kₘ rather than the Vₘₐₓ of the kinase reaction. Competitive inhibition is well-understood in enzymology, and the consequences of competitive inhibition need not be recited herein. Typically, compounds screened as positive are involved in hydrogen-bonding interactions with residues Lys181, Ala232, Thr292, and Thr162 (FIG. 3D) similar to interactions observed in the crystal structure of Akt1 in complex with AMP-PNP. Therefore, in one preferred alternative, another screening step is performed, that of measuring a consensus between scoring patterns and hydrogen bonding patterns substantially similar to that observed in the crystal structure of Akt1 in complex with AMP-PMP and selecting compounds that exhibit both highly ranked scoring patterns and hydrogen bonding patterns substantially similar to that observed in the crystal structure of Akt1 in complex with AMP-PMP. This measurement of the consensus improves the hit rate of the overall screening process substantially.

The compounds to be selected can be from any suitable library of small molecule compounds. One library is obtainable from Chembridge (San Diego, Calif.). Other libraries are available, and methods for their preparation are described, for examples in R. B. Silverman, “The Organic. Chemistry of Drug Design and Drug Action” (2d ed., Elsevier. Amsterdam), pp. 41-43, incorporated herein by this reference. Scaffolds for synthesis can be derived, for example, from natural products.

Among the compounds having Akt1 inhibitory activity are Compounds 1 and 2 (Table 1), showing IC₅₀ values in the low-micromolar range. Compound 3 had an IC₅₀ of 25.1 μM.

In addition, Compounds 4 and 5 (Table 2), which are derivatives of Compound 1, have limited inhibitory activity against Akt1 kinase.

Accordingly, another aspect of the present invention is a method of derivatizing a compound determined to have inhibitory activity against Akt1 kinase to improve its inhibitory activity comprising the steps of:

(1) providing a compound having inhibitory activity against Akt1 kinase;

(2) derivatizing the compound by introducing at least one covalent modification thereto to produce at least one derivative; and

(3) screening the derivatives produced in step (2) for inhibitory activity against Akt1 kinase; and

(4) selecting a derivative that has improved inhibitory activity against Akt1 kinase as compared with the compound provided in step (1).

The derivatization can include one or more reactions well known in organic chemistry and in the art of drug design, including the substitution of halogens for one or more hydrogens and the replacement of halogens by hydrogens, the placement, removal or repositioning of carboxyl groups on aromatic rings, the conversion of carboxylic acids into esters and vice versa, the conversion of alcohols into ethers, the substitution of hydrogens on amine groups with alkyl groups or the removal of alkyl groups on amine groups, and other similar reactions. The derivatization can be carried out under standard reaction conditions employing reagents well known in the art, such as those disclosed in M. B. Smith & J. March, “March's Advanced Organic Chemistry: Reactions, Mechanisms, and Structure (5^{th} ed., John Wiley & Sons, New York, 2001), incorporated herein by this reference. Other derivatization reactions can be used.

Accordingly, another aspect of the invention is a pharmaceutical composition for inhibiting Akt 1 kinase comprising:

(1) a compound whose activity in inhibiting Akt1 kinase was discovered by the screening method described above in a quantity sufficient to inhibit Akt1 kinase; and

(2) a pharmaceutically acceptable carrier.

Typically, the compound has an IC₅₀ of less than about 100 μM. Preferably, the compound has an IC₅₀ of less than about 30 μM. More preferably, the compound has an IC₅₀ of less than about 10 μM. Still more preferably, the compound has an IC₅₀ of less than about 5 μM.

The pharmaceutical composition can be formulated for the treatment of cancer or for the treatment of another condition characterized by dysregulation of apoptosis, including neurodegenerative conditions.

Among preferred compounds for the preparation of pharmaceutical compositions are Compounds 1, 2, 3, 4, and 5. Among particularly preferred compounds for the preparation of pharmaceutical compositions are Compounds 1 and 2, so that the compound is selected from the group consisting of Compound 1 of formula (I), Compound 2 of formula (II), Compound 3 of formula (III), and Compounds 4 and 5 of formula (IV), where, in formula IV, for Compound 4, R is p-COOH and for Compound 5, R is m-COOH,

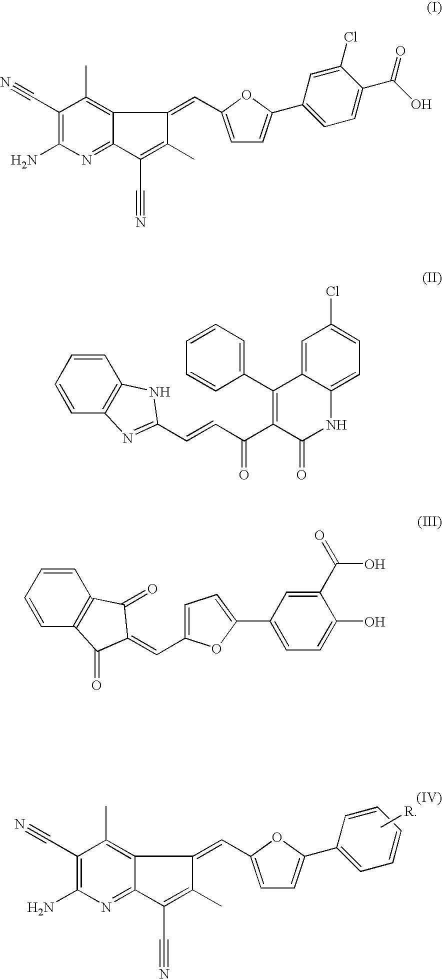

Toxicity and therapeutic efficacy of compounds in pharmaceutical compositions according to the present invention can be determined by standard pharmaceutical procedures in cell cultures or experimental animals, e.g. for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

For any compound used in the pharmaceutical compounds according to the present invention, the therapeutically effective dose can be estimated initially from cell culture assays. For example, a dose can be formulated in animal models to achieve a circulating plasma concentration range that includes the IC₅₀ as determined in cell culture (i.e., the concentration of the test compound which achieves a half-maximal improvement in receptor signaling when chronic effects are considered). Such information can be used to more accurately determine useful doses in humans. Levels in plasma may be measured, for example, by HPLC.

The exact formulation, route of administration and dosage for pharmaceutical compositions according to the present invention can be chosen by the individual physician in view of the patient's condition. (See e.g. Fingi et al., in The Pharmacological Basis of Therapeutics, 1975, Ch. 1 p. 1. It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administered dose in the management of the disorder of interest wilt vary with the severity of the condition to be treated and to the route of administration. The severity of the condition may, for example, be evaluated, in part, by standard prognostic evaluation methods. Further, the dose and perhaps the dose frequency, will also vary according to the age, body weight, and response of the individual patient. A program comparable to that discussed above may be used in veterinary medicine.

Depending on the specific conditions being treated, such pharmaceutical compositions may be formulated and administered systemically or locally. Typically, administration is systemic. Techniques for formulation and administration may be found in Remington's Pharmaceutical Sciences, 18th ed., Mack Publishing Co., Easton, Pa. (1990). Suitable routes may include oral, rectal, transdermal, vaginal, transmucosal, or intestinal administration, parenteral delivery, including intramuscular, subcutaneous, intramedullary injections, as well as intrathecal, direct intraventricular, intravenous, intraperitoneal, intranasal, or intraocular injections, just to name a few. Typically, oral administration is preferred

For injection, the pharmaceutical compositions of the invention may be formulated in aqueous solutions. For such transmucosal administration, penetrants appropriate to the barrier to be permeated are used in the formulation. Such penetrants are generally known in the art.

Use of pharmaceutically acceptable carriers is within the scope of the invention. For example, for oral administration, carriers are well known in the art. Such carriers enable the compounds of the invention to be formulated as tablets, pills, capsules, liquids, gels, syrups, slurries, suspensions and the like, for oral ingestion by a patient to be treated.

In addition to the active ingredients, such as the compound with Akt1 kinase inhibition activity, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methylcellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent, mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Accordingly, another aspect of the invention is a method of treating a disease or condition characterized by dysregulation of apoptosis comprising administering an effective quantity of a pharmaceutical composition according to the present invention to a subject diagnosed with or suspected of having a disease or condition characterized by dysregulation of apoptosis in order to normalize apoptosis. The disease or condition is typically cancer, but can be a neurodegenerative condition. The subject diagnosed with or suspected of having the disease or condition can be human, but, alternatively, can be a socially or economically important animal selected from the group consisting of a dog, a cat, a sheep, a horse, a cow, a pig, a goat, a chicken, a turkey, a duck, a goose, and any other eukaryote. Apoptosis is a universal process in cell regulation of eukaryotes.

TABLE 1
Structures of Compounds Showing Inhibitory Activity Against Akt1

| Molecule | ID | Structure | IC₅₀ (μM) | Ki (μM) |
|---|---|---|---|---|
| 1 | 6025233 | 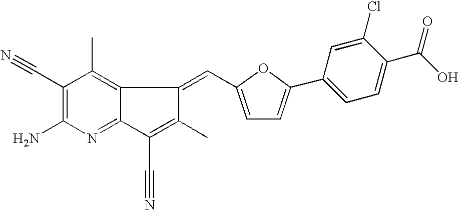 | 2.6 | 1.1 |
| 2 | 5809365 | 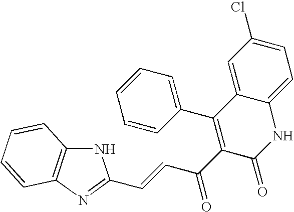 | 4.5 | 3.9 |
| 3 | 5378650 | 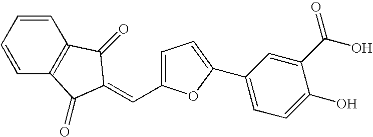 | 25.1 | 20.8 |

TABLE 2
Derivatives of Compound 1 and Their Inhibitory Activity Against Akt1

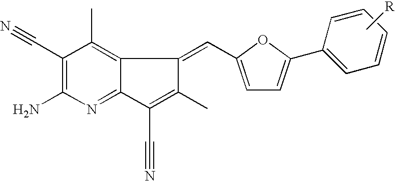

| Compound | R | IC₅₀ | |
|---|---|---|---|
| 1 | para-COOH, meta-Cl | 2.6 | μM |
| 4 | para-COOH | 126 | μM |
| 5 | meta-COOH | 60.2 | μM |

The invention is illustrated by the following Example. This Example is included for illustrative purposes only, and is not intended to limit the invention.

### EXAMPLE

Nowadays, high-throughput screening of large chemical databases is a common approach for lead identification. However given the 3D structure of the protein target, it should be possible to restrict the number of compounds to be tested by using computational docking studies.

In this Example, we describe a number of approaches based on the reported crystal structure of Akt1 kinase. This methodology allowed us to select several potential inhibitors on the basis of their predicted ability of docking into the ATP binding site.

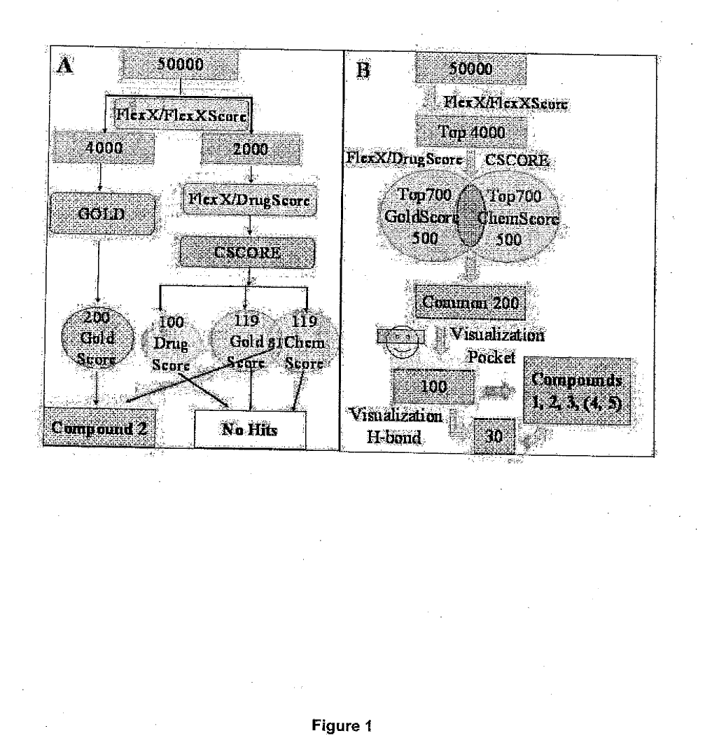

A target binding site was derived from the crystal structure of the ternary complex involving Akt1 non-hydrolyzable form of ATP (AMP-PNP pdb id: 1O6K) and the peptide-substrate derived from GSK-3β (10). The protein active site was defined including those residues within 6.5 Å from the ATP mimic. Hydrogen atoms were calculated using Sybyl (11)((Tripos, St. Louis, Mo.) and water molecules, peptide substrate as well as the ATP mimic were eliminated. 50000 compounds (Chembridge San Diego, Calif., USA) were subsequently docked and ranked according to the software FlexX (BioSolveIT, Sankt Augustin, Germany) (12, 13). In an initial attempt, we selected the top 2000 compounds and ranked them with other scoring functions using CSCORE (14) (Sybyl). Subsequently, we experimentally tested at 10 μM the top 100 compounds according to Drugscore (15), the top 200 compounds according to Goldscore (16) and another top 200 compounds according to Chemscore (17). Disappointingly, only one inhibitor (compound 2, 5809365) common in Goldscore and Chemscore selection was found through the Akt1 assay (Table 1), while no inhibitor was found among compounds selected by Drugscore. In addition, we also docked the FlexX top 4000 compounds using GOLD (18) and subsequently selected and tested the top 200 compounds. Once again, compound 2 resulted as the only inhibitor (FIG. 1A). FIG. 1 shows a schematic representation of the virtual docking approaches adopted.

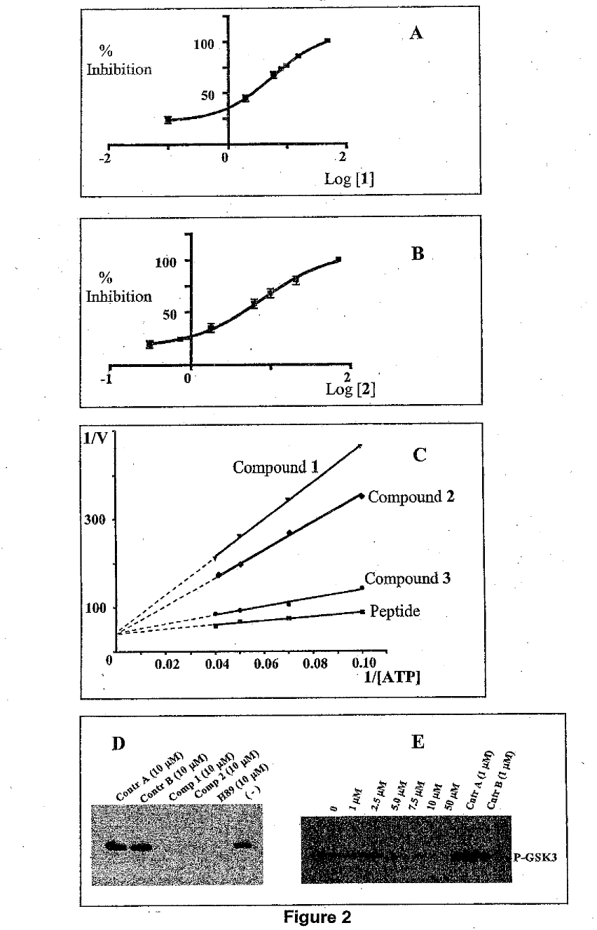

Based on these results, we relied on another strategy described in FIG. 1B. Here the top 4000 compounds out of 50000 docked compounds were selected using FlexX and Drugscore (BioSolvIT). The top 4000 docked structures were further evaluated and ranked according to Goldscore and Chemscore functions (CSCORE). A list of common 200 compounds was then selected among ranked top 700 compounds according to both scoring functions (FIG. 1B). Visual analysis of the 200 docked structures resulted in the elimination of 100 compounds with improbable docking geometry. The remaining 100 compounds were experimentally tested up to 30 μM against Akt1. The inhibitory activity was evaluated for the selected compounds by using Z′-LYTE™ kit assay provided by Invitrogen Corporation (19). Among the experimentally tested compounds at least three emerged as interesting inhibitors, two of which showing IC₅₀ values in the low micromolar range. Particularly, compounds 1 and 2 (Table 1) inhibited Akt1 in a concentration range comparable to that of H-89, the only known commercially available Akt inhibitor (20), yielding IC₅₀ values of 2.3 μM and 4.5 μM, respectively (FIG. 2 A-B). Compound 3 showed an IC₅₀ value of 25.1 μM. Remaining selected compounds did not show any inhibitory activity up to 30 μM. In FIG. 2: A) IC₅₀ evaluation for compound 1 (2.6 μM). The Hill slope for this curve is 1.1; B) IC₅₀ evaluation for compound 2 (4.5 μM). Corning®384-well low volume plates (20 μl) were used. The fluorescent-enzymatic assay has been performed following the protocol provided by Invitrogen Corporation, using a fluorescent plate reader (Victor2, Perkin-Elmer). IC₅₀ values were determined fitting the data to the sigmoidal dose/response equation and plotting the observed percentage of inhibition versus the logarithm of inhibitor concentration using GraphPad Prism®. C) Lineweaver-Burk Kₘ and Kₘ(app) evaluation for Akt1. Each measurement was performed in triplicate. The Kₘ and Vₘₐₓ values of the enzymatic reaction were determined at 25° C. by using increasing ATP concentrations (5, 10, 15, 20 and 25 μM). The Kᵢ and the Kₘ(app) were calculated at fixed inhibitor concentrations, as reported in the text. All constant values were definitely evaluated by fitting the data to the Lineweaver-Burk plot; D) Akt1 inhibition assay using GSK-3 as a substrate. Comparison of compounds 1 and 2 (Table 1) with H89 at 10 μM. E) Dose response for compound 1. Akt (10 ng of recombinant enzyme) in 25 μl 1X kinase buffer (25 mM Tris, pH 7.5; 5 mM β-glycerol phosphate; 2 mM dithiothreitol; 0.1 mM Na₃VO₄; and 10 mM MgCl, was mixed with 2.5 μl DMSO (1% stock) or MPA-D (100 μM in 1% DMSO). Samples were incubated on ice for 1.5 hours at which time 1 μg of GSK-3 fusion protein (Colt Signaling), which served as the substrate, was added followed by ATP (200 μM) to each reaction mixture. After the suspensions were incubated at 30° C. for 20 min, the reaction was terminated by the addition of 3×SDS sample buffer (187.5 mM Tris-HCl, pH 6.8; 6% SDS; 30% glycerol; 150 mM dithiothreitol; and 0.03% bromophenol blue). The samples were boiled for 5 min, and the proteins were separated on a 12% SDS polyacrylamide gel and subsequently transferred to a nitrocellulose membrane. Membranes were incubated with rabbit polyclonal anti-phospho-GSK-3α/β (Ser21/9) (Cell Signaling).

A second assay was carried out, in order to further evaluate the inhibitory activity for compounds 1 and 2 by using an immuno-blotting assay with anti-phospho-GSK-3α/β and GSK-3 as a substrate (FIG. 2 D-E). In agreement with the Z′-LYTE™ assay, both compounds inhibited GS3K phosphorylation in the low micromolar range.

To confirm and extend these findings, we measured the Kᵢ value and the type of inhibition of Akt1 by compounds 1, 2 and 3 (FIG. 2C). For these purposes, we initially determined the Kₘ and the Vₘₐₓ of the enzymatic reaction involving the peptide provided by the Z′-LYTE™ kit assay and Akt1 by varying the concentration of ATP. The above parameters appeared to be 7.9 μM and 0.0205 μmol min⁻¹ mg⁻¹, respectively. We then used a 10 μM concentration of compound 1, 20 μM of compound 2 and 50 μM of compound 3, in order to identify the inhibitors' Kᵢ values (Table 1). Because all our inhibitors affected the Kₘ rather than the Vₘₐₓ of the reaction (FIG. 2C), they can be truly considered ATP-competitive inhibitors of Akt1.

To rule out the possibility of eventual non-specific interactions, we also verified that no substantial changes in the IC₅₀ values for compounds 1 were detected, when increasing 10 fold the protein concentration as well as by pre-incubating the compounds with Akt1 for 30 minutes prior measuring its CO₅₀ value. These simple tests have been shown to give dramatically different 1050 values in presence of non-specific ligand-protein interactions (21).

In addition, we tested our compounds against a non related protein kinase, Abl1 (22), which is a tyrosine kinase under investigation in our laboratory. Our compounds did not inhibit this kinase at concentrations up to 100 μM. We currently do not have data on the selectivity of our compounds for the different Akt isoforms.

Therefore using our structure-based approach we were able to identify three inhibitors of Akt1 (Table 1), two of which showed an inhibitory activity comparable to that of H-89 (FIG. 2). Based on the docked geometry, and in agreement with our experimental data, it appears that all three inhibitors place themselves nicely into the catalytic site of the ATP, resembling the binding of the adenosine moiety of the cofactor (FIG. 3 A,B,C). Indeed each compound is involved in H-bonding interactions with residues Lys181, Ala232, Thr292 and Thr162 (FIG. 3D) similarly to what observed in the crystal structure of Akt1 in complex with AMP-PNP. In FIG. 3, A), B) and C) Docked structures of compounds 1, 2 and 3 into the ATP binding site of Akt1. The 2D structures of 50000 compounds were converted to 3D structures using CONCORD (25) or CORINA (26). Two docking programs were used to screen compounds against Akt1 kinase. FlexX program applied Drugscore to determine docked conformers. GOLD package docked ligands using Goldscore fitness function. Consensus scoring was obtained by using CSCORE (Sybyl). D) Hydrogen-bonds between compound 1 and residues present in the Akt1 catalytic pocket.

Accordingly, measurement of inhibitory properties of additional 13 analogues of compound 1 revealed that only compounds 4 and 5, both capable of forming H-bonds with the above mentioned residues, showed appreciable inhibition in the micromolar range (Table 2).

Therefore the ability of a given compound to be involved in H-bond interaction appears to be essential in all the inhibitor-Akt1 complexes as previously reported for other protein kinases (23) and in other docking studies (27). In fact, when the ability of forming H-bonds is taken into account for the selection of candidate inhibitors, only 30 compounds would be selected. As described in FIG. 1B, our selected 30 compounds contain all three hits thus yielding a hit rate of 10%.

Despite the availability of many reliable in silico approaches and robust in vitro commercially available assays, discovering Akt inhibitors still remains a challenging task. Even though several attempts have been performed in this field, there are presently no marketable inhibitors against Akt1 besides H-89. In fact, a very recent paper reports on a study, which based on high-throughput screening, led to the characterization of only two Akt1 low micromolar inhibitors out of 270,000 tested compounds (23).

Moreover, during our ongoing efforts to identify molecules capable of blocking Akt-kinases activity, we also tested a library of 2000 natural products (Microsource) at a concentration up to 30 μM against Akt1, but no compound emerged as an effective low micromolar inhibitor (data not shown).

In conclusion, in the present Example we describe two different structure-based strategies we adopted in order to discover compounds inhibiting Akt1. When we applied the strategy described in FIG. 1A, simply relying on results provided by the scoring functions, very disappointingly the hit rate appeared to be only slightly superior to the one expected from a random approach (0.01-0.5%) (24). However, when our docking methodology outcome was analyzed by taking into account a consensus between scoring functions and H-bonding patterns similar to those observed in the crystal structure of Akt1 in complex with AMP-PNP, a remarkable 10% hit rate was finally achieved (FIG. 1B).

We believe that the two low micromolar inhibitors described here may represent a starting point for finding potent and selective molecules capable of preventing Akt1 activity in human tumoral cells.

### REFERENCES

The following references are cited in the specification and Example by reference number; all of these references are incorporated by this reference in the application in their entirety:
1. Klingmuller, U. The role of tyrosine phosphorylation in proliferation and maturation of erythroid progenitor cells—signals emanating from the erythropoietin receptor. Eur. J. Biochem. 1997, 249, 637-647.
2. Lev, D. C.; Kim, L. S.; Melnikova, V.; Ruiz, M.; Ananthaswamy, H. N.; Price, J. E. Dual blockade of EGFR and ERK1/2 phosphoylation potentiates growth inhibition of breast cancer cells. Brit. J. of Cancer 2004, 91, 795-802
3. Dickson, L. M.; Rhodes, C. J. Pancreatic beta-cell growth and survival in the onset of type 2 diabetes: a role for protein kinase B in the Akt? Am. J. Physiol. Endocrinol. Metab. 2004, 287, 192-198.
4. Okano, J.; Gaslightwala, I.; Birnbaum, M. J.; Rustgi, A. K.; Nakagawa, H. Akt/protein kinase β isoforms are differentially regulated by epidermal growth factor stimulation. J. Biol. Chem. 2000, 275, 30934-33942.
5. Chijiwa, T.; Mishima, A.; Hagiwara, M.; Sano, M.; Hayashi, K.; Inoue, T.; Naito, K.; Toshioka, T.; Hidaka H. Inhibition of forskolin-induced neurite outgrowth and protein phosphorylation by a newly synthesized selective inhibitor of cyclic AMP-dependent protein kinase, N-[2-(p-bromocinnamylamino)ethyl]-5-isoquinolinesulfonamide (H-89), of PC12D pheochromocytoma cells. J. Biol. Chem. 1990, 265, 5267-5272.
6. Stratford, S., Hoehn, K. L., Liu, F., and Summers, S. A. Regulation of insulin action by ceramide: dual mechanisms linking ceramide accumulation to the inhibition of Akt/protein kinase B. J. Biol. Chem. 2004, 279, 36608-36615.
7. Baxter, C. A.; Murray, C. W.; Waszkowycz, B.; Li, J.; Sykes, R. A.; Bone, R. G. A.; Perkins, T. D. J.; Wylie, W. New approach to molecular docking and its application to virtual screening of chemical databases. J. Chem. Inf. Comput. Sci. 2000, 40, 254-262.
8. Carry, R.; Jhoti, H. Structure-based screening of low-affinity compounds. Drug Discov. Today 2002, 7, 522-527.
9. Perola, E.; Xu, K.; Kollmeyer, T. M.; Kaufmann, S. H.; Prendergast, F. G.; Pang, Y. P. Successful Virtual Screening of a Chemical Database for Farnesyltransferase Inhibitor Leads. J. Med. Chem., 2000, 43, 401-408.
10. Yang, J.; Cron, P.; Good, V. M.; Thompson, V.; Hemmings, B. A.; Barford, D. Crystal Structure of an Activated Akt/Protein Kinase B Ternary Complex with Gsk-3 Peptide and AMP-PNP. Nat. Struct. Biol. 2002, 9, 940.
11. SYBYL. 6.9. 1699 South Hanley Road, St. Louis, Mo., 63144, USA: Tripos Inc.
12. Rarey, M.; Kramer, B.; Lengauer, T.; Klebe, G. A fast flexible docking method using an incremental construction algorithm. J. Mol. Biol 1996, 261, 470-489.
13. FlexX 1.13.5. Sankt Augustin, Germany BioSolveIT GmbH.
14. Clark, R. D.; Strizhev, A.; Leonard, J. M.; Blake, J. F.; Matthew, J. B. Consensus scoring for ligand/protein interactions. J. Mol. Graph. Model. 2002, 20, 281-295
15. Gohlke, H.; Hendlich, M.; Klebe, G. Knowledge-based Scoring Function to Predict Protein-Ligand Interactions. J. Mol. Biol 2000, 295, 337-356.
16. Jones, G.; Willett, P.; Glen, R. C.; Leach, A. R.; Taylor, R. Development and validation of a genetic algorithm for flexible docking. J. Mol. Biol. 1997, 267, 727-748.
17. Eldridge, M. D.; Murray, C. W.; Auton, T. R.; Paolini, G. V.; Mee, R. P. Empirical scoring functions: 1. The development of a fast empirical scoring function to estimate the binding affinity of ligands in receptor complexes. J. Comput-Aided Mot Des. 1997, 11, 425-445.
18. GOLD. 2.1. 12 Union Road, Cambridge, CB2 1EZ, UK: The Cambridge Crystallographic Data Centre.
19. Rodems, S.; A. H amman, B. D.; Lin, C.; Zhao, J.; Shah, S.; Heidary, D.; Makings, L.; Stack, J. H.; Pollok, B. A. A FRET-Based Assay Platform for Ultra-High Density Drug Screening of Protein Kinases and Phosphatases. ASSAY and Drug Development Technologies 2001, 1.
20. Reuveni, H.; Livnah, N.; Geiger, T.; Klein, S.; Ohne, O.; Cohen, I.; Benhar, M.; Gellerman, G.; Levitzki, A. Toward a PKB inhibitor: modification of a selective PKA inhibitor by rational design. Biochemistry 2002, 41, 10304-10314.
21. McGovern et al., J. Med. Chem. 45, 1712-22 (2002).
22. Daub, H.; Specht, K; Ullrich, A. Strategies to overcome resistance to targeted protein kinase inhibitors. Nature reviews, 2004, 3, 1001-1010.
23. Barnet, S. F.; Defeo-Jones, D.; Fu, S.; Hancock, P. J.; Haskell, K. M.; Jones, R. E.; Kahana, J. A.; Kral, A. M.; Leander, K.; Lee, L. L.; Malinowski, J.; McAvoy, E. M.; Nahas, D. D.; Robinson, R. G.; Huber, H. E. Identification and characterization of pleckstrin homology domain dependent and isozyme specific Akt inhibitors. Biochemical Journal Immediate Publication. Published on 29 Sep. 2004 as manuscript BJ20041140.
24. Doman, T. N.; et al. Molecular docking and high-throughput screening for novel inhibitors of protein tyrosine phosphatase-1B. J. Med. Chem. 2002, 45, 2213-2221.
25. Pearlman, R. S. Rapid Generation of High Quality Approximate 3-dimension Molecular Structures. Chem. Des. Auto. News, 1987, 2, 1.
26. Sadowski, J.; Gasteiger, J.; Klebe, G. J. Comparison of Automatic Three-Dimensional Model Builders Using 639 X-ray Structures. Chem. Inf. Comput. Sci. 1994, 34, 1000-1008.
27. Examination of docked ligand orientation sampling methodology; mapping a pharmacophore for success. J. Mol. Graph, Model. 2003, 22(1), 31-40.

### ADVANTAGES OF THE INVENTION

The present invention provides a rapid, efficient method for screening large numbers of compounds for inhibitory activity for protein kinase B, a critical enzyme in controlling apoptosis and other cellular functions. Because of the use of docking models by the method, the method has a higher hit rate than random screening. Compounds determined to have inhibitory activity by screening methods according to the present invention are likely to be useful in treating cancer and other conditions characterized by dysregulation of apoptosis, and pharmaceutical compositions including such compounds can be prepared.

The inventions illustratively described herein can suitably be practiced in the absence of any element or elements, limitation or limitations, not specifically disclosed herein. Thus, for example, the terms “comprising,” “including,” containing, etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

We claim:

1. A method of screening compounds for inhibition of Akt1 kinase activity comprising the steps of:
(a) providing a plurality of compounds suspected of having Akt1 kinase inhibitory activity;
(b) modeling the docking of each of the plurality of the compounds with a target binding site derived from the crystal structure of a ternary complex involving Akt1, a nonhydrolyzable ATP analogue, and a peptide substrate derived from a physiological AKT substrate such that the protein active site is defined including those residues within a defined distance from the nonhydrolyzable ATP analogue;
(c) ranking the docked compounds by goodness of fit;
(d) further selecting compounds from compounds high ranked by goodness of fit in docking by using one or more screening criteria;
(e) optionally, visually analyzing structures of compounds selected in step (d) to remove any compounds with improbable docking geometry; and
(f) experimentally testing the selected compounds from step (d) or step (e), if step (e) is performed, to determine their inhibitory activity against Akt1 in order to select compounds with Akt1 inhibitory activity.

2. The method of claim 1 wherein the nonhydrolyzable ATP analogue is AMP-PNP.

3. The method of claim 1 wherein the peptide substrate is a peptide substrate derived from GSK-3β.

4. The method of claim 1 wherein the defined distance from the nonhydrolyzable analogue is from about 6.0 Å to about 7.0 Å.

5. The method of claim 4 wherein the defined distance from the nonhydrolyzable analogue is about 6.5 Å.

6. The method of claim 1 wherein the modeling of docking is performed using a docking algorithm.

7. The method of claim 6 wherein the docking algorithm is FlexX.

8. The method of claim 1 wherein the step of further selecting compounds from compounds high ranked by goodness of fit in docking by using one or more screening criteria is performed by using one or more of CSCORE (SYBYL), Drugscore, Goldscore, Chemscore, and GOLD.

9. The method of claim 6 wherein the step of further selecting compounds from compounds high ranked by goodness of fit in docking by using one or more screening criteria is performed by first using Drugscore, and then evaluating and ranking the top docked structures according to Goldscore and Chemscore individually.

10. The method of claim 9 wherein compounds that are highly ranked according to both Goldscore and Chemscore functions, when those are applied individually, are then selected for visual analysis to remove compounds with improbable docking geometries.

11. The method of claim 1 wherein the step of experimentally testing the compounds that emerge from screening in step (d) or step (e), if performed, is performed by testing the compounds at a concentration up to 30 μM.

12. The method of claim 11 wherein the concentration is 10 μM.

13. The method of claim 1 wherein compounds screened as positive are capable of binding specifically within the catalytic site of the ATP.

14. The method of claim 1 wherein compounds screened as positive act as competitive inhibitors of Akt1, competing with ATP.

15. The method of claim 1 wherein compounds screened as positive are involved in hydrogen-bonding interactions with residues Lys181, Ala232, Thr292, and Thr162 of Akt1.

16. The method of claim 1 further comprising an additional screening step of measuring a consensus between scoring patterns and hydrogen bonding patterns substantially similar to that observed in the crystal structure of Akt1 in complex with AMP-PMP and selecting compounds that exhibit both highly ranked scoring patterns and hydrogen bonding patterns substantially similar to that observed in the crystal structure of Akt1 in complex with AMP-PMP.

17. A method of derivatizing a compound determined to have inhibitory activity against Akt1 kinase to improve its inhibitory activity comprising the steps of:
(a) providing a compound having inhibitory activity against Akt1 kinase;
(b) derivatizing the compound by introducing at least one covalent modification thereto to produce at least one derivative; and
(c) screening the derivatives produced in step (b) for inhibitory activity against Akt1 kinase; and
(d) selecting a derivative that has improved inhibitory activity against Akt1 kinase as compared with the compound provided in step (a).

18. The method of claim 17 wherein the step of derivatizing comprises at least one reaction selected from the group consisting of the substitution of halogens for one or more hydrogens; the replacement of halogens by hydrogens; the placement, removal or repositioning of carboxyl groups on aromatic rings; the conversion of carboxylic acids into esters and vice versa; the conversion of alcohols into ethers; the substitution of hydrogens on amine groups with alkyl groups; and the removal of alkyl groups on amine groups.

19. A pharmaceutical composition for inhibiting Akt 1 kinase comprising:
(a) a compound whose activity in inhibiting Akt1 kinase was discovered by the screening method of claim 1 in a quantity sufficient to inhibit Akt1 kinase; and
(b) a pharmaceutically acceptable carrier.

20. The pharmaceutical composition of claim 19 wherein the compound has an IC₅₀ of less than about 100 μM.

21. The pharmaceutical composition of claim 20 wherein the compound has an IC₅₀ of less than about 30 μM.

22. The pharmaceutical composition of claim 21 wherein the compound has an IC₅₀ of less than about 10 μM.

23. The pharmaceutical composition of claim 22 wherein the compound has an IC₅₀ of less than about 5 μM.

24. The pharmaceutical composition of claim 19 wherein the pharmaceutical composition is formulated for the treatment of cancer.

25. The pharmaceutical composition of claim 19 wherein the pharmaceutical composition is formulated for the treatment of a condition characterized by the dysregulation of apoptosis other than cancer.

26. The pharmaceutical composition of claim 25 wherein the pharmaceutical composition is formulated for the treatment of a neurodegenerative condition.

27. The pharmaceutical composition of claim 19 wherein the a compound whose activity in inhibiting Akt1 kinase was discovered by the screening method is selected from the group consisting of Compound 1 of formula (I) Compound 2 of formula (II), Compound 3 of formula (III), and Compounds 4 and 5 of formula (IV), where, in formula IV, for Compound 4, R is p-COOH and for Compound 5, R is m-COOH

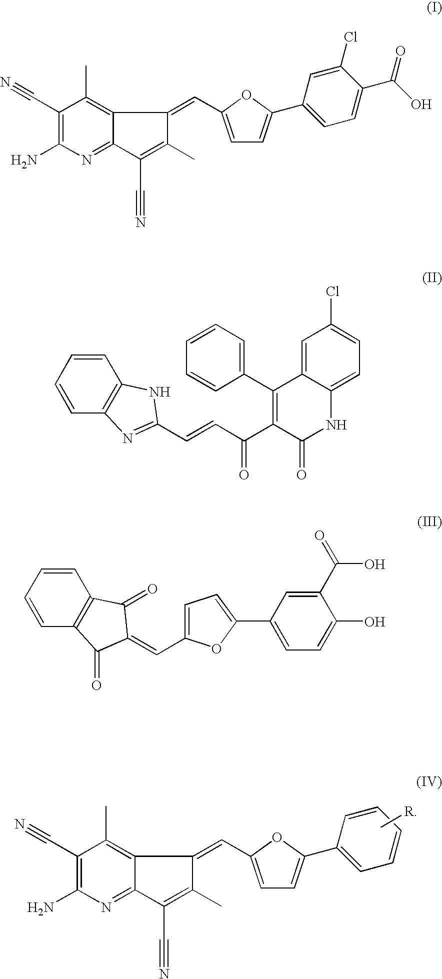

28. A method of treating a disease or condition characterized by dysregulation of apoptosis comprising administering an effective quantity of the pharmaceutical composition of claim 19 to a subject diagnosed with or suspected of having a disease or condition characterized by dysregulation of apoptosis in order to normalize apoptosis.

29. The method of claim 28 wherein the disease or condition characterized by dysregulation of apoptosis is cancer.

30. The method of claim 28 wherein the disease or condition characterized by dysregulation of apoptosis is a neurodegenerative condition.

31. The method of claim 28 wherein the subject is human.

32. The method of claim 28 wherein the subject is a socially or economically important animal selected from the group consisting of a dog, a cat, a sheep, a horse, a cow, a pig, a goat, a chicken, a turkey, a duck, and a goose.
